(19) United States
(12) Patent Application Publication
(10) Pub. No.: US 2011/0098684 A1
(43) Pub. Date: Apr. 28, 2011

(54) CATHETER DEVICE FOR THE CONTROL OF LIQUID DISCHARGE FROM THE URINARY BLADDER BY A USER PERSON

(76) Inventor: Antoine Trubiano, Montreal (CA)

(21) Appl. No.: 12/999,385

(22) PCT Filed: Jun. 15, 2009
(86) PCT No.: PCT/CA2009/000839

§ 371 Date: Dec. 16, 2010

(30) Foreign Application Priority Data
Jun. 17, 2008 (CA) 2,635,157
Jun. 8, 2009 (CA) 2,668,299

(51) Int. Cl. A61M 27/00

(52) U.S. Cl. 604/544

(57) ABSTRACT

A catheter device for the control of liquid discharge from the urinary bladder, and its use, is described. The catheter device is an elongated flexible tube of elastic material having an inlet opening at a distal end thereof and one or more inflatable balloons secured about the tube spaced from the inlet opening. The balloon retains the catheter at a predetermined position within the urethra while preventing urine to flow about the catheter below the balloon. The tube is provided with a numbered scale printed in a predetermined proximal end portion of the tube to provide a visual indication to a user person of the length of the catheter inserted in the urethra to permit the positioning of the one or more balloons at a desired location in the urethra below the bladder during repetitive removal and insertion of the catheter.

### TECHNICAL FIELD

The present invention relates to a catheter device for the control, by a user person, of liquid discharge from the urinary bladder through the urethra of a person.

### BACKGROUND ART

Urinary incontinence is defined as the accidental leakage of urine through the urethra. Prostate problems and post radical prostatectomy urinary incontinence greatly affects a male's quality of life. The National Institute of Diabetes and Digestive and Kidney Diseases (NIDDK) has reported that urinary incontinence is a medical problem and that there are four forms of urinary incontinence. These are (1) temporary and reversible incontinence related to urinary track infection, constipation or delirium; (2) stress incontinence caused by weak pelvic and sphincter muscles; (3) urgent continence caused by damaged or iritatable nerves; and (4) overflow incontinence that results when an individual is unable to empty the bladder.

Various methods and devices have been developed to try and treat these problems. One such treatment is to insert a catheter through the urethra to drain the bladder. The catheter then leads to a bag in which the fluid from the bladder is collected. A major problem with these catheters is that they often develop infections and stone formation not to mention the discomfort of carrying and emptying a bag with urine on a regular basis. They also require frequent disinfecting and cleaning. Cauterization is usually done by a doctor but a patient may be easily trained to effect the procedure himself. To do this, there is a need to learn sterile techniques to avoid urinary track infections.

A more recent technique is to use an artificial sphincter which is implanted adjacent the urethra below the bladder to keep the urethra closed until it is time to urinate. As reported in medical publications, this device can help people who have incontinence because of weak sphincter muscles or because of nerve damage that interferes with sphincter muscle function. It does not solve incontinence caused by uncontrolled bladder contraction. Artificial sphincters consist of a cup that fits around the urethra with a small balloon reservoir placed in the abdomen and a pump placed in the scrotum. The cup is filled with a liquid that makes it fit tightly around the urethra to squeeze the urethra to prevent urine from leaking. When it is time to urinate the user squeezes the pump with the fingers to deflate the cup so that the liquid moves to the balloon reservoir from the cuff and urine can now flow through the urethra. When the bladder is emptied, the cup automatically refills within a time delay of about 2 to 5 minutes to keep the urethra tightly closed. This solution has not been found to work efficiently and requires interaction with the user to release the urine.

In recent years a new procedure has been developed to treat urinary incontinence. This new procedure comprises implanting a balloon which is connected to a conduit tube with the conduit tube remaining inside a person's body and the balloon is positioned adjacent the urethra whereby upon inflation of the balloon, through the scrotum, the urethra will be squeezed and hopefully close. The tube is provided with an inlet port positioned in the scrotum and through which a fluid is injected by a syringe, through the skin of the scrotum, whereby to inject a proper amount of fluid in the balloon to expand it to apply sufficient pressure against the urethra to close it. This technique has also encountered various problems, and it has been reported that the success rate is no better than fifty percent (50%). A major problem with this technique is that the urethra is unstable and when pressure is applied against it the urethra will be displaced in an uncontrollable manner. The balloons are also unstable. This is why the efficiency rate has not been satisfactory. Usually there are two of these balloons that are implanted one on opposed sides of the urethra and sometimes offset from one another. Reference to U.S. Pat. Nos. 6,045,498 and 6,645,138 describes such implantable devices and their operation.

As reported in Medical News Today, Newsletter dated Oct. 24, 2006, these balloons are implanted beneath the bladder neck to increase its resistance. The novel difference with this device is the ability to adjust the tightness of the urethral occlusion by altering the amount to fluid in each balloon via a titanium port connector that can be accessed via a percutaneous injection in the scrotum. A study of this technique is also reported in the May 2006 issue of Urology. With this technique balloon adjustment is required to achieve continence and the average number of adjustments was 4.6, all of which were done in an out patient setting and in first six months after placement. A revision surgery was also required in four of twenty-three patients.

The above-mentioned technique appears to be on course to eventually resolve problems associated with balloon implants. However, there is still a need to resolve major problems with this technique such as the assurance that the implant will effectively engage the urethra and effect proper closure thereof by applying a pressure customized to the patient's needs depending on his degree of control to evacuate urine from the bladder. Another problem to be resolved is the implantation of the device itself about the urethra to effectively assure the proper function thereof prior to closing the incision.

It has also been reported by NIDDK that women experience incontinence twice as often as men. Pregnancy and childbirth, menopause, and the structure of the female urinary tract account for this difference. But both women and men can become incontinent from neurologic injury, birth defects, strokes, multiple sclerosis and physical problems associated with aging. Incontinence in women usually occurs because of problems with muscles that help to hold or release urine.

Many types of treatments are used to treat incontinence in women, depending in the severity of their problem, such as exercises, electrical stimulation, biofeedback timed voiding or bladder training, medications, pessaries, implants, surgery and catherization.

With the above-mentioned urinary problems where there is uncontrolled retention of urine in the bladder, the bladder will eventually shrink due to its inability to retain liquid as the liquid substantially flows through the bladder and into a bag outside the user's body. The disadvantages of using a bag are discussed hereinabove. The present invention resolves the problem of having to carry a bag to act as a bladder and this is achieved by using a catheter device wherein the person's bladder acts as the bag and is discharged when the user experiences the normal sensation to do so.

### DISCLOSURE OF INVENTION

It is a feature of the present invention to provide a catheter device for the control of liquid discharge from the urinary bladder and wherein the catheter has an inlet opening at a distal end thereof and one or more inflatable balloons spaced from said inlet opening and positioned within the urethra and spaced below the bladder. The catheter has a discharge conduit which is in communication with the inlet opening and is closed at a discharge end thereof by a closure means which permits liquid accumulated in the bladder to be discharged by the removal of the closure means and directed in conventional urinals, toilets, etc.

Another feature of the present invention is to provide a catheter device comprised of an elongated flexible tube of elastic material and wherein the tube has position indicator means provided in a predetermined proximal end portion thereof to provide a visual indication to a user person of the length of the catheter inserted in the urethra to permit the positioning of the balloon at a desired location below the urinary bladder during repetitive insertion and removal of the catheter.

Another feature of the present invention is to provide the method of use of the catheter device of the present invention.

According to a broad aspect of the present invention there is provided a catheter device for the control of liquid discharge from the urinary bladder through the urethra. The catheter device comprises an elongated flexible tube of elastic material. The tube has an inlet opening at a distal end thereof communicating with a discharge conduit of the tube. One or more inflatable balloons are secured about the tube and has an opening communicating with a channel extending in a side wall of the tube. The conduit and the channel are secured to a fluid port hub connected at a proximal end of the tube. The hub has a fluid valve port communicating with the channel and a discharge port communicating with the discharge conduit. Closure means removably closes the discharge port. The fluid valve port is adapted to receive a needle of a syringe in sealing engagement therewith to admit and retrieve fluid in the balloon wherein to inflate or deflate the balloon. The tube has position indicator means provided in a predetermined proximal end portion thereof to provide a visual indication to a user person of the length of the catheter inserted in the urethra to permit the positioning of the balloon at a desired location below the urinary bladder during repetitive insertion of the catheter.

According to a further broad aspect there is a single elongated inflatable balloon secured about the tube.

According to another broad aspect there are at least three inflatable balloons secured about the tube.

According to a further broad aspect of the present invention there is provided the use of a fluid discharge catheter for the control of liquid discharge from the urinary bladder of a person.

According to a further broad aspect of the present invention there is provided the method of controlling liquid discharge from the urinary bladder of a person by a catheter device constructed in accordance with the present invention and positionable at a predetermined location within the urethra of the user person below the urinary bladder.

### BRIEF DESCRIPTION OF DRAWINGS

A preferred embodiment of the present invention will now be described with reference to the accompanying drawings in which:

FIG. 1 is a schematic illustration of the catheter device of the present invention positioned in the urethra of a male organ;

FIG. 2 is an enlarged view of a portion of FIG. 1;

FIG. 3 is a fragmented view illustrating the construction of the distal end portion of the catheter device;

FIG. 4 is a schematic view of the proximal end portion of the catheter flexible tube showing a valve operated discharge arrangement;

FIG. 5A is a fragmented perspective view illustrating how the bladder is evacuated by the use of the catheter device;

FIG. 5B is an assembly view illustrating a further version of the catheter device;

FIG. 6 is a schematic illustration of the catheter device of the present invention provided with an elongated balloon and positioned in the urethra of a male organ;

FIG. 7 is a schematic illustration of the catheter device of the present invention provided with at least three balloons and positioned in the urethra of a male organ; and

FIG. 8 is an enlarged fragmented and partly section view showing the fluid channel communicating with the balloons.

### MODES FOR CARRYING OUT THE INVENTION

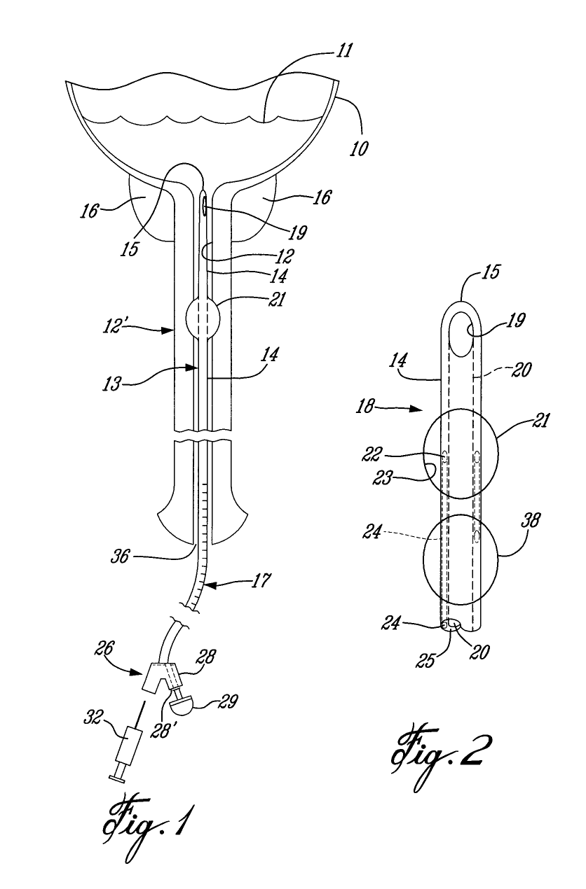

Referring now to the drawings, and more particularly to FIG. 1, there is shown in schematic illustration the bladder 10 of a person adapted to retain urine 11 therein. The urethra 12 communicates with the bladder 10 to discharge urine therefrom. As hereinshown, the catheter device 13 of the present invention is comprised of an elongated flexible tube 14 which is inserted in the urethra 12 of the male organ 12′ at a predetermined location and spaced below the bladder 10. As hereinshown, the distal end 15 of the catheter device 13 is positioned below the prostate glands 16 and a proximal end section 17 extends out of the urethra to provide hand manipulation of this section by a user person to direct urine flow into toilets or urinals.

Referring to FIG. 2, there is shown the distal end portion 18 of the catheter device 13. As can be seen, the elongated flexible tube 14 has an inlet opening 19 which communicates with a discharge conduit 20 extending along the tube 14. The tube is formed of a suitable flexible elastic material, such as silicone, capable of being disinfected and suitable for medical use. An inflatable balloon 21 is secured about the flexible tube 14 and an opening 22 formed in the tube. The opening 22 communicates the inner chamber 23 of the inflatable balloon 21 with a channel 24 extending in a side wall 25 of the tube.

With further reference to FIG. 4 there is shown the proximal end portion of the catheter device 13. It can be seen that the tube 14 is connected to a fluid port hub 26 which is sealingly connected at a proximal end of the tube. The hub 26 has a fluid valve port 27 communicating with the channel 24. It also has a discharge port 28 communicating with the discharge conduit 20 through a valve housing 30. The valve housing 30 is provided with a finger operable valve element 30′ to position the valve to an open position, as shown, or to a closed position 30″, as shown in phantom lines. A discharge conduit 20′ is used by the user to direct urine for disposal.

As shown in FIG. 1, the closure means is in the form of a plug 29 which is removably positionable in the free end opening 28′ of the discharge port to open and close off the discharge port to permit the evacuation of urine liquid from the bladder 10.

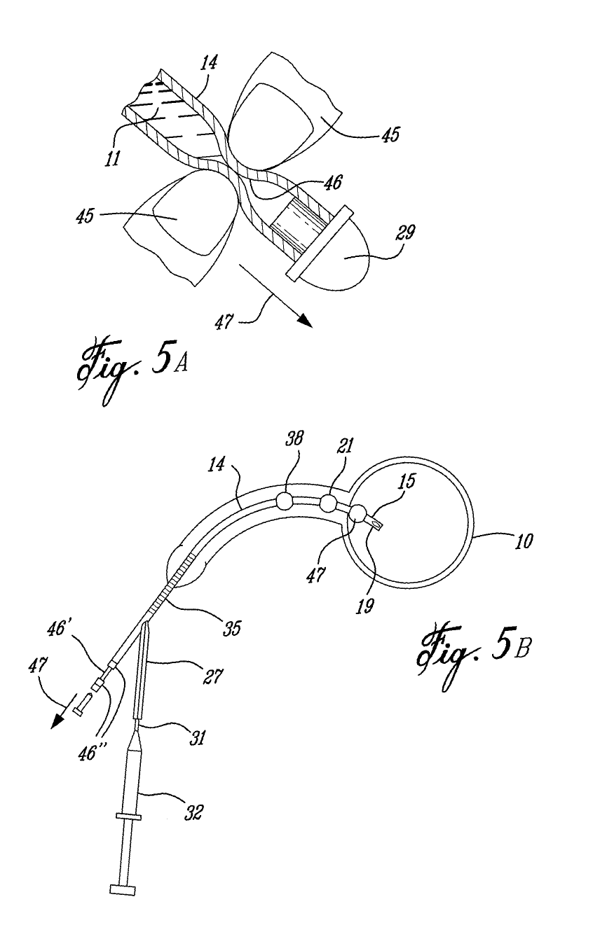

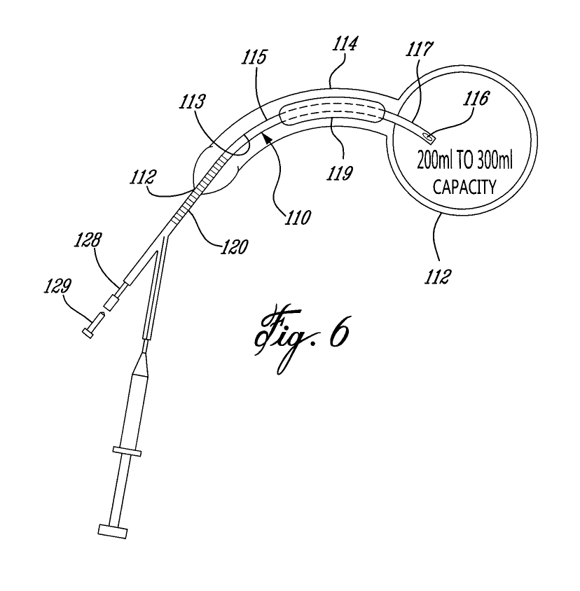

The fluid valve port 27, see also FIG. 5B, is adapted to receive a needle 31 of a syringe 32 to admit and retrieve fluid from the inflatable balloon 21 wherein to inflate or deflate the balloon to retain and remove the catheter from the urethra. The balloon is also used to prevent discharge of urine from the bladder around the catheter below the balloon.

As shown in FIGS. 1 and 2, because the inflatable balloon is inflated in the urethra this can be very painful as the urethra passage in the male organ 12′ is very sensitive, but with time, if the balloon is positioned substantially always at the same position, the tissue surrounding the urethra, at a predetermined location, will expand and adapt to pressure exerted by the balloon upon expansion and become less sensitive. Of course, the balloon is injected a predetermined amount of liquid, herein about 8 cc, such that it always expands to substantially the same size. However, to position a urethra with the balloon at substantially a precise location in the urethra, without visual access, is difficult and to resolve this problem the catheter of the present invention is provided with position indicator means in the form of a numbered scale 35 which is printed on the elongated flexible tube along its proximal end section 17. By visually observing the scale, a user person can position the distal end and the inflatable balloon of the catheter at a substantially predetermined location within the urethra spaced from the outer end 36 of the urethra where the scale is observed. Accordingly, the balloon is always at the same location in the urethra where the surrounding tissue has adapted to expansion by the balloon. This is important as such a catheter needs to be periodically removed from the urethra for disinfection and cleaning and repositioned therein or a new urethra repositioned therein. By repetitive positioning of the balloon at substantially the same location, the surrounding tissues adapt to the inflated balloon resulting in substantially painless installation, as opposed to inflating the balloon at a different location where the surrounding tissue has not undergone pressure.

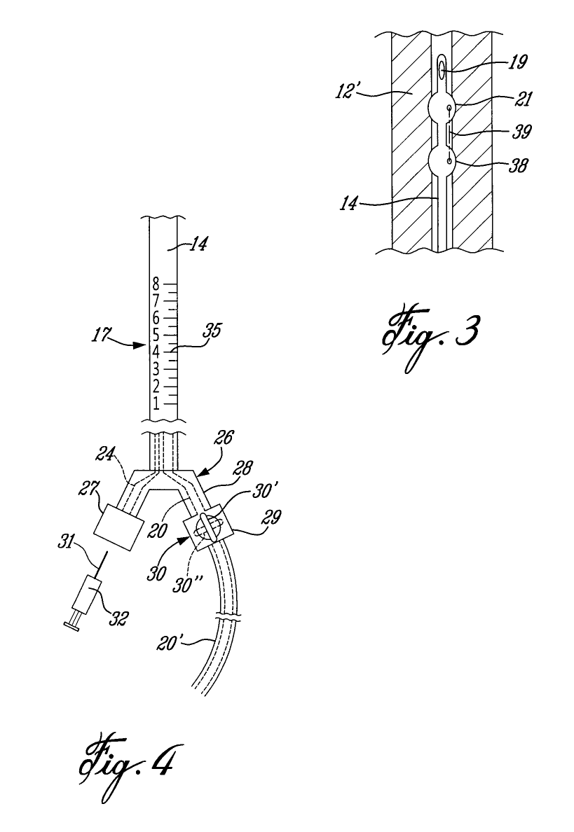

FIG. 3 illustrates a modification of the catheter of the present invention wherein a second inflatable balloon 38 is shown secured about the flexible tube 14 and spaced below the first inflatable balloon 21. An interconnecting channel 39 interconnects the balloons together to admit and remove fluid therefrom. Reference is made to U.S. Pat. No. 6,167,886 which shows the interconnection of two spaced-apart balloons about a catheter. The reason for two balloons is that it requires less fluid in each balloon, about 4 cc, to retain the catheter securely in position. Accordingly, there is less pressure exerted on the walls of the urethra making the application less painful and preventing leakage about the catheter when positioned in the urethra.

It is pointed out that in the method of use when the catheter is first inserted in the urethra it will cause the bladder, which has shrunk, to start accumulating liquid and consequently expand. After many repetitive uses, the bladder will expand to retain more and more urine therein and returning to its intended function. Accordingly, the bladder can now be considered as a substitute for the bag which was usually connected to the discharge port 28 of the flexible tube.

Below is a chart illustrating the effective use of the bladder to accumulate urine when a catheter of the present invention is secured in the urethra. This chart illustrates the volume retained in the bladder between certain time periods from May 7 to May 12, 2008. As the bladder fills up, it creates an urge in the person to evacuate the bladder and now this is easily done in a conventional manner simply by positioning the proximal end section 17 of the tube and pointing the discharge port towards a urinal or toilet and by removing the plug 29 the fluid from the bladder is discharged through the catheter device 13 of the present invention.

CHART

TIME

| DATE | From | To | VOLUME |
|---|---|---|---|
| May 7-8, 2008 | 10:00 p.m. | 1:30 a.m. | 500 ml |
| May 8, 2008 | 1:30 a.m. | 7:15 a.m. | 550 ml |
| May 8, 2008 | 7:15 a.m. | 12:30 p.m. | 175 ml |
| May 8, 2008 | 12:30 p.m. | 4:00 p.m. | 125 ml |
| May 8, 2008 | 4:00 p.m. | 6:30 p.m. | 75 ml |
| May 8, 2008 | 6:30 p.m. | 8:30 p.m. | 175 ml |
| May 8, 2008 | 8:30 p.m. | 10:15 p.m. | 150 ml |
| May 8-9, 2008 | 10:15 p.m. | 12:45 a.m. | 600 ml |
| May 9, 2008 | 12:45 a.m. | 3:15 a.m. | 510 ml |
| May 9, 2008 | 3:15 a.m. | 7:45 a.m. | 505 ml |
| May 9, 2008 | 7:45 a.m. | 11:30 a.m. | 150 ml |
| May 9, 2008 | 11:30 a.m. | 2:15 p.m. | 175 ml |
| May 9, 2008 | 2:15 p.m. | 5:30 p.m. | 125 ml |
| May 9, 2008 | 5:30 p.m. | 8:45 p.m. | 100 ml |
| May 9, 2008 | 8:45 p.m. | 12:00 a.m. | 75 ml |
| May 10, 2008 | 12:00 a.m. | 1:45 a.m. | 350 ml |
| May 10, 2008 | 1:45 a.m. | 5:45 a.m. | 500 ml |
| May 10, 2008 | 5:45 a.m. | 7:30 a.m. | 110 ml |
| May 10, 2008 | 7:30 a.m. | 11:00 a.m. | 75 ml |
| May 10, 2008 | 11:00 a.m. | 4:00 p.m. | 175 ml |
| May 10, 2008 | 4:00 p.m. | 8:00 p.m. | 150 ml |
| May 10, 2008 | 8:00 p.m. | 11:00 p.m. | 75 ml |
| May 10-11, 2008 | 11:00 p.m. | 6:30 a.m. | 350 ml |
| May 11, 2008 | 6:30 a.m. | 7:45 a.m. | 75 ml |
| May 11, 2008 | 7:45 a.m. | 12:30 p.m. | 175 ml |
| May 11, 2008 | 12:30 p.m. | 4:00 p.m. | 210 ml |
| May 11, 2008 | 4:00 p.m. | 6:30 p.m. | 110 ml |
| May 11-12, 2008 | 6:30 p.m. | 12:30 a.m. | 150 ml |
| May 12, 2008 | 12:30 a.m. | 6:30 a.m. | 375 ml |
| May 12, 2008 | 6:30 a.m. | 8:30 a.m. | 175 ml |

FIG. 5A illustrates the method of use of the catheter device to evacuate urine 11 from the bladder 10. When the wearer person needs to evacuate urine, an end portion of the flexible tube 14 is pinched between the fingers 45 to temporarily cause a blockage 46 in the tube 14 adjacent the plug 29 or the hub 26. The plug is then removed by a pulling force with the other hand in the direction of arrow 47. The pinched blockage is then released and the urine flows out of the tube 14. After evacuation, the plug 29 is reinserted. If a valve is used in the hub 26 instead of a plug, then the valve is merely opened and closed to evacuate urine.

FIG. 5B illustrates further modifications wherein the area to be punched is delineated by a tube area 46′ of reduced diameter to indicate to the wearer a precise area to pinch immediately above the plug to minimize accidental leakage of urine when the plug is removed. Alternatively, the area to be pinched may be indicated by spaced bands 46″ which are color printed on the tube 14. With this means of identification, there would be no need to have a reduced diameter in the tube 14. As hereinshown, the catheter device 13 is also provided with a third balloon 47 to retain the distal end 15 of the catheter tube 14 in the bladder 10, as is conventional in the art with catheters having a collection bag at its proximal end.

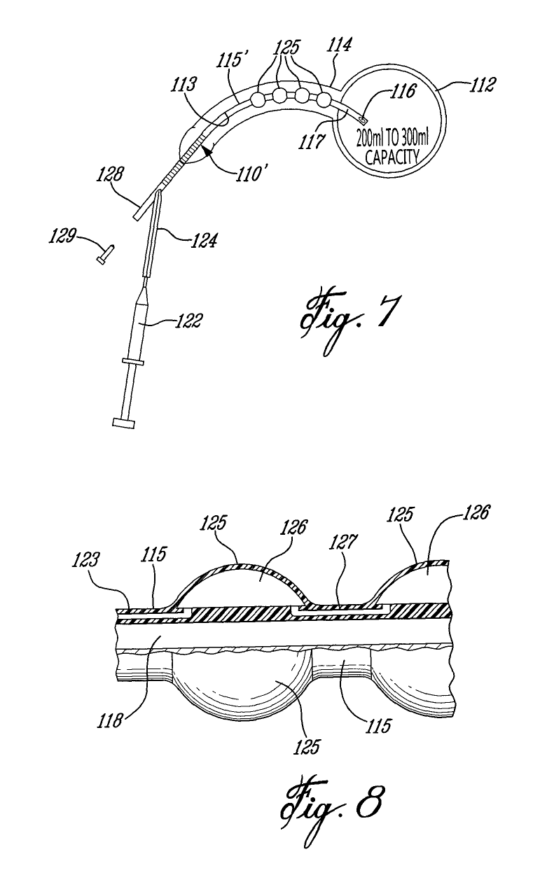

FIG. 6 shows a further embodiment of the catheter device 110 of the present invention adapted for the control of liquid discharge form the urinary bladder 112 through the urethra 113 of a male organ 114. FIG. 7 shows a still further embodiment of the catheter device 110′ of the present invention to effect the same function. The catheter device 110 comprises an elongated flexible tube 115 formed of suitable elastic material as is well known in the art. The elongated flexible tube 115 has an inlet opening 116 at a distal end 117 thereof which communicates with a discharge conduit 118 (see FIG. 8) of the tube. An elongated inflatable balloon 119 is secured about the tube 115 and has an opening communicating with a channel extending in the side wall of the tube. This opening and channel are illustrated in FIG. 8 with respect to the embodiment of the catheter device 110′ but the elongated inflatable balloon 119 is also constructed to communicate with the channel which is adapted to receive a fluid therein for expanding the elongated inflatable balloon 119.

The catheter device 110 is provided with the numbered scale 120 in a portion thereof whereby a user person can position the distal end 117 and the inflatable balloon 119 of the catheter at a substantially predetermined location within the urethra 113 and spaced from the outer end 121 of the urethra where the scale is observed. Accordingly, the distal end may always be positioned in the urinary bladder 112 and the elongated balloon at a substantially precise location within the urethra where the surrounding tissue has adapted to expansion by the balloon.

Referring to FIG. 7, there is shown a further modification of the urethra 110′ of the present invention and as hereinshown a plurality of balloons 125, herein at least three, are secured about the elongated flexible tube 115′ and in a spaced-apart arrangement. Such an arrangement provides an enhanced retention of the catheter within the urethra 113 against the pressure of the urine accumulated in the bladder and prevents leakage of urine about the balloons. The surface texture of the balloons 21, 38, 119 and 125 may be ribbed or roughened for better adherence once inflated.

As previously described, in order to inflate the balloons 125 a needle syringe 122 injects a fluid within the fluid port 124 which is in communication with a fluid channel 123 extending within the wall of the flexible tube 115, as is illustrated in FIGS. 7 and 8. The fluid channel 123 is in communication with the inner area 126 of the balloons 125. A secondary fluid channel 127 interconnects each of the balloons 125 to one another as shown in FIG. 8. In the embodiment of FIG. 6, the channel 123 communicates with the inner area of the balloon 119 (not shown) but in a similar fashion as illustrated in FIG. 6 with respect to the balloons 125.

The discharge conduit 118, see FIG. 8, which communicates with the urinary bladder 112 has a discharge end 128 which is adapted to receive a plug 129 which constitutes a closure means, as previously described. When the wearer of the catheter device senses an urge to evacuate urine from the bladder the discharge end 128 is positioned over a recipient, such as a toilet or urinal and the plug 129 is removed to discharge the urine from the bladder 112.

It is within the ambit of the present invention to cover any obvious modifications of the catheter device of the present invention as above described, provided such modifications fall within the scope of the appended claims. For example, the position indicator means is hereinshown as a numbered scale but other position indicating means can be foreseen. It is also pointed out that the use of the valve housing 30 prevents the accidental loss of a plug. The combination of both a valve and plug is also foreseen.

1. A catheter device for the control of liquid discharge from the urinary bladder through the urethra, said catheter device comprising an elongated flexible tube of elastic material, said tube having an inlet opening at a distal end thereof communicating with a discharge conduit of said tube, one or more elongated inflatable balloons secured about said tube and having an opening communicating with a channel extending in a side wall of said tube, said discharge conduit having a discharge end, said channel being secured to a fluid port hub connected at a proximal end of said channel, said hub having a fluid valve port communicating with said channel, closure means to removably close said discharge end of said discharge conduit, said fluid valve port being adapted to receive a needle of a syringe in sealing engagement therewith to admit and retrieve fluid in said elongated balloon wherein to inflate or deflate said balloon.

2. The catheter device of claim 1 wherein there is further provided position indicator means in a predetermined proximal end portion of said tube to provide a visual indication to a user person of the length of said catheter inserted in the urethra to permit the positioning of said elongated inflatable balloon at a desired location below said urinary bladder during repetitive insertion of said catheter.

3. The catheter device as claimed in claim 2 wherein said position indicator means is a measurement scale printed on said elongated flexible tube.

4. The catheter device as claimed in claim 1 wherein said closure means is a plug removably secured in said discharge end of said discharge conduit.

5. A catheter device as claimed in claim 2 wherein said position indicator means is a measurement scale printed on said elongated flexible tube.

6. A catheter device as claimed in claim 1 wherein there is further provided a second inflatable balloon secured about said tube spaced from said inflatable balloon, and an interconnecting channel interconnecting said balloon together to admit and remove fluid therefrom.

7. A catheter device as claimed in claim 1 wherein there are at least three inflatable balloons secured spaced-apart about said tube and each having an opening communicating with a channel between said balloons and a main channel extending in a side wall of said tube to admit and remove fluid therefrom.

8. A catheter device as claimed in claim 1 wherein said one elongated inflatable balloon is an elongated inflatable balloon.

9. The use of a fluid discharge catheter for the control of liquid discharge from the urinary bladder of a person, said catheter having an elongated flexible tube of elastic material adapted to be positioned in the urethra of said person, said tube having an inlet opening at a distal end thereof for receiving liquid from said urinary bladder, a discharge conduit in said tube in communication with said inlet opening and having a removable closure means at a proximal end thereof, one or more inflatable balloons secured about said tube and having an opening communicating with a channel extending in a side wall of said tube, a fluid injectable in said balloon through said channel to inflate said balloon to arrest said catheter in said urethra at a substantially predetermined location below said urinary bladder which is determined by a visual position indicator means provided in a predetermined proximal end portion of said elongated flexible tube whereby said inlet opening and said one or more balloons can be positioned at said predetermined location below said urinary bladder during respective insertion of said catheter from said urethra.

10. The use of the catheter of claim 9 wherein said predetermined location is determined by said position indicator means being a visual numbered scale.

11. The use of the catheter of claim 10 wherein said elongated flexible tube has a hand-manipulatable proximal end section to permit access to said hand-manipulatable proximal end section by the user person for the removal of said removable closure means and the positioning of said proximal end section to evacuate liquid from said urinary bladder.

12. The use of the catheter of claim 11 wherein the closure means is one of a plug or a valve retained at a discharge end of said tube.
